(19) United States
(12) Patent Application Publication
(10) Pub. No.: US 2015/0241412 A1
(43) Pub. Date: Aug. 27, 2015

(54) SELF-ASSEMBLED NANOSTRUCTURED SENSORS AND METHODS THEREOF

(71) Applicant: AUBURN UNIVERSITY, Auburn, AL (US)

(72) Inventors: Marko HAKOVIRTA, Opelika, AL (US); Burak AKSOY, Auburn, AL (US)

(21) Appl. No.: 13/837,013

(22) Filed: Mar. 15, 2013

Related U.S. Application Data
(60) Provisional application No. 61/727,948, filed on Nov. 19, 2012.
(60) Provisional application No. 61/723,141, filed on Nov. 6, 2012.
(60) Provisional application No. 61/672,940, filed on Jul. 18, 2012.

(51) Int. Cl. G01N 33/52; C12Q 1/46; C12Q 1/10; G01N 33/12

(52) U.S. Cl. CPC G01N 33/521; G01N 33/12; C12Q 1/46; C12Q 1/10; G01N 2458/30

(57) ABSTRACT

The present disclosure provides compositions comprising a diatom and a sensor, including gas detecting compositions, bacteria detecting compositions, explosive degradation product detecting compositions, and neurotoxin detecting compositions. The disclosure also provides methods of identifying the presence of gas, bacteria, explosive degradation products, and neurotoxins utilizing the compositions.

### CROSS-REFERENCE TO RELATED APPLICATIONS

This application claims the benefit under 35 USC §119(e) of U.S. Provisional Application Ser. No. 61/672,940, filed on Jul. 18, 2012, U.S. Provisional Application Ser. No. 61/723,141, filed on Nov. 6, 2012, and U.S. Provisional Application Ser. No. 61/727,948, filed on Nov. 19, 2012, the entire disclosures of which are incorporated herein by reference.

### TECHNICAL FIELD

The invention relates to self-assembled nanostructured diatom sensor compositions comprising a diatom and a sensor. The invention includes compositions and methods utilizing the compositions for detection of gases, bacteria, explosive degradation products, and/or neurotoxins.

### BACKGROUND AND SUMMARY OF THE INVENTION

Effective detection of potentially hazardous and harmful substances on products and in the environment is a significant goal for numerous industries. For example, detecting the spoilage or contamination of food products before their consumption by consumers is critical for maintaining safety in the food industry. In addition, the rapid detection of bacteria, neurotoxins, and explosives is important for many essential fields, including preservation of industrial safety, healthcare environments, and national security.

However, the rapid and accurate detection of substances in such varied and complex settings is difficult to achieve. Presently utilized methods are subject to undesirably low sensitivity to identify such substances, which could potentially be harmful. Furthermore, current methods may have an undesirably short time frame for effectiveness. Moreover, presently available procedures may be extremely expensive to manufacture or may require highly trained and skilled operators to accomplish the procedure, thus limiting their use and adaptability in the various settings.

Therefore, there exists a need for new compositions and methods that provide highly sensitive and rapid detection of substances in a variety of settings. In addition, new compositions and methods utilizing an inexpensive, long-lasting, and adaptable detection system of substances are also very desirable. Accordingly, the present disclosure provides nanostructured diatom sensor compositions and methods of using the compositions, which exhibit desirable properties and provide related advantages for improvement in the detection of many different substances which could potentially be hazardous and harmful.

The present disclosure provides compositions comprising a diatom and a sensor, including gas detecting compositions, bacteria detecting compositions, explosive degradation product detecting compositions, and neurotoxin detecting compositions. The disclosure also provides methods of identifying the presence of gas, bacteria, explosive degradation products, and neurotoxins utilizing the compositions.

The compositions and methods utilizing a diatom and a sensor according to the present disclosure provide several advantages compared to other compositions and methods known in the art. First, the compositions have a greatly improved sensitivity for identification compared to currently available products. For example, in food safety applications, the compositions of the present disclosure comprising pH-sensitive dyes can detect food spoilage such as nitrogen compounds at an order of approximately 5 parts per million (ppm). Consequently, the compositions of the present disclosure have a superior sensitivity that is approximately three orders of magnitude greater than the sensitivity findings generated by other researchers.

Second, the compositions of the present disclosure are able to provide sustained or prolonged shelf life compared to other products known in the art. For example, the activity of enzyme sensors in the compositions demonstrate fully preserved activity after one month of storage. The compositions can have a shelf life of several months without the need for special packaging (e.g., film wrapping), and the shelf life can potentially be extended to one year or more with use of special packaging.

Third, the compositions of the present disclosure comprise diatoms, which are an abundant natural material that can be obtained and produced at a lower cost compared to currently available technologies. Diatoms are available at various grades, representing a number of different sizes and surface areas, and can be used inexpensively to produce the disclosed compositions for detection of substances, including those which are potentially hazardous and harmful. In comparison, materials used to produce currently available detection products utilizing carbon nanotubes are between 9 and 900 times more expensive. Moreover, the compositions of the present disclosure advantageously have a reduced carbon foot print compared to detection products utilizing carbon nanotubes.

Fourth, the compositions of the present disclosure provide are able to provide rapid detection of substances compared to other processes in the art. For instance, in food spoilage applications, many current techniques are designed as laboratory quality control measures and are not used in-situ due to their complexity and cost. The most commonly used food spoilage detection method analyzes total viable counts (TVC) of bacteria and/or specific spoilage bacteria, which requires an incubation period of one to three days for colony formation. In comparison, the compositions of the present disclosure provide can be utilized to obtain detection results on a more expedited basis, and is based on visible and distinct color changes of the sensor that can be easily detected. Moreover, the color change of the sensor may be visible for hours or even days, allowing for evaluation on an extended basis.

Finally, the compositions of the present disclosure are adaptable to traditional systems (e.g., traditional paper making processes), possess desirable properties for scalability and reversibility, and may be non-invasive with respect to the products from which substance detection is desired.

The following numbered embodiments are contemplated and are non-limiting:

1. A composition comprising a diatom and a sensor.

2. The composition of clause 1, wherein the diatom is a diatom frustule.

3. The composition of clause 1, wherein the diatom is a diatomite.

4. The composition of clause 1, wherein the diatom is diatomaceous earth.

5. The composition of any one of clauses 1 to 4, wherein the surface of the diatom comprises one or more pores.

6. The composition of clause 5, wherein at least part of the sensor is contained within one or more pores in the diatom.

7. The composition of any one of clauses 1 to 6, wherein the sensor is an organic reagent.

8. The composition of any one of clauses 1 to 6, wherein the sensor is an inorganic reagent.

9. The composition of any one of clauses 1 to 6, wherein the sensor is a dye.

10. The composition of any one of clauses 1 to 6, wherein the sensor is an enzyme.

11. The composition of any one of clauses 1 to 6, wherein the sensor is a salt.

12. The composition of clause 11, wherein the salt is an XG salt.

13. The composition of any one of clauses 1 to 6, wherein the sensor is a fluorescent agent.

14. The composition of any one of clauses 1 to 6, wherein the sensor is DNA.

15. The composition of any one of clauses 1 to 6, wherein the sensor is a protein.

16. The composition of any one of clauses 1 to 6, wherein the sensor is an antibody.

17. The composition of any one of clauses 1 to 6, wherein the sensor is a quantum dot.

18. The composition of any one of clauses 1 to 16, wherein the sensor changes color following exposure to a gas.

19. The composition of clause 17, wherein the gas is a nitrogen-containing gas.

20. The composition of clause 17 or clause 18, wherein the gas is a total volatile basic nitrogen (TVB-N) gas.

21. The composition of any one of clauses 17 to 19, wherein the gas is ammonia.

22. The composition of any one of clauses 17 to 20, wherein the sensor changes color following exposure to volatile basic nitrogen.

23. The composition of any one of clauses 1 to 16, wherein the sensor changes color following a change in pH.

24. The composition of any one of clauses 1 to 16, wherein the sensor changes color following a change in temperature.

25. The composition of any one of clauses 1 to 16, wherein the sensor changes color following exposure to a neurotoxin.

26. The composition of clause 24, wherein the neurotoxin is an acetylcholinesterase inhibitor.

27. The composition of any one of clauses 1 to 16, wherein the sensor changes color following exposure to bacteria.

28. The composition of clause 26, wherein the bacteria is an airborne bacteria.

29. The composition of any one of clauses 1 to 16, wherein the sensor changes color following exposure to an enzyme.

30. The composition of any one of clauses 1 to 16, wherein the sensor changes color following exposure to an explosive degradation product.

31. The composition of clause 29, wherein the explosive degradation product is nitrogen.

32. The composition of clause 29, wherein the explosive degradation product is ammonia.

33. The composition of any one of clauses 1 to 31, wherein the composition further comprises an article, and wherein the diatom is applied to the article.

34. The composition of clause 32, wherein the diatom is applied to the article using a polymer binder.

35. The composition of clause 32 or clause 33, wherein the article is paper.

36. The composition of clause 34, wherein the diatom is applied to the paper using inkjet printing.

37. The composition of clause 34 or clause 35, wherein the paper is a sticker.

38. The composition of clause 34 or clause 35, wherein the paper is a label.

39. The composition of any one of clauses 34 to 37, wherein the paper comprises packaging for a food product.

40. The composition of clause 32 or clause 33, wherein the article is packaging.

41. The composition of clause 39, wherein the diatom is applied to the packaging using inkjet printing.

42. The composition of clause 39 or clause 40, wherein the packaging is for a food product.

43. The composition of clause 32 or clause 33, wherein the article is a tissue towel.

44. The composition of clause 32 or clause 33, wherein the article is a nonwoven.

45. The composition of clause 32 or clause 33, wherein the article is a gas permeable coating.

46. The composition of clause 32 or clause 33, wherein the article is a gas permeable paint.

47. The composition of clause 32 or clause 33, wherein the article is a storage wall.

48. The composition of clause 32 or clause 33, wherein the article is a filter.

49. The composition of clause 32 or clause 33, wherein the article is a felt tip.

50. A gas detecting composition comprising a diatom and a sensor.

51. The gas detecting composition of clause 50, wherein the diatom is a diatom frustule.

52. The gas detecting composition of clause 50, wherein the diatom is a diatomite.

53. The gas detecting composition of clause 50, wherein the diatom is diatomaceous earth.

54. The gas detecting composition of any one of clauses 50 to 53, wherein the surface of the diatom comprises one or more pores.

55. The gas detecting composition of clause 54, wherein at least part of the sensor is contained within one or more pores in the diatom.

56. The gas detecting composition of any one of clauses 50 to 55, wherein the sensor is an organic reagent.

57. The gas detecting composition of any one of clauses 50 to 55, wherein the sensor is an inorganic reagent.

58. The gas detecting composition of any one of clauses 50 to 55, wherein the sensor is a dye.

59. The gas detecting composition of any one of clauses 50 to 55, wherein the sensor is an enzyme.

60. The gas detecting composition of any one of clauses 50 to 55, wherein the sensor is a salt.

61. The gas detecting composition of clause 60, wherein the salt is an XG salt.

62. The gas detecting composition of any one of clauses 50 to 55, wherein the sensor is a fluorescent agent.

63. The gas detecting composition of any one of clauses 50 to 55, wherein the sensor is DNA.

64. The gas detecting composition of any one of clauses 50 to 55, wherein the sensor is a protein.

65. The gas detecting composition of any one of clauses 50 to 55, wherein the sensor is an antibody.

66. The gas detecting composition of any one of clauses 50 to 55, wherein the sensor is a quantum dot.

67. The gas detecting composition of any one of clauses 50 to 66, wherein the sensor changes color following exposure to a gas.

68. The gas detecting composition of clause 67, wherein the gas is a nitrogen-containing gas.

69. The gas detecting composition of clause 67, wherein the gas is a total volatile basic nitrogen (TVB-N) gas.

70. The gas detecting composition of any one of clauses 67 to 69, wherein the gas is ammonia.

71. The gas detecting composition of any one of clauses 50 to 66, wherein the sensor changes color following exposure to volatile basic nitrogen.

72. The gas detecting composition of any one of clauses 50 to 66, wherein the sensor changes color following a change in pH.

73. The gas detecting composition of any one of clauses 50 to 66, wherein the sensor changes color following a change in temperature.

74. A gas detecting system comprising the gas detecting composition of clause 50 and an article.

75. The gas detecting system of clause 74, wherein the gas detecting composition is applied to the article.

76. The gas detecting system of clause 74, wherein the gas detecting composition is applied to the article using a polymer binder.

77. The gas detecting system of any one of clauses 74 to 76, wherein the article is paper.

78. The gas detecting system of clause 77, wherein the gas detecting composition is applied to the paper using inkjet printing.

79. The gas detecting system of clause 77 or 78, wherein the paper is a sticker.

80. The gas detecting system of clause 77 or 78, wherein the paper is a label.

81. The gas detecting system of any one of clauses 77 to 80, wherein the paper comprises packaging for a food product.

82. The gas detecting system of any one of clauses 74 to 76, wherein the article is packaging.

83. The gas detecting system of clause 82, wherein the diatom is applied to the packaging using inkjet printing.

84. The gas detecting system of any one of clauses 82 to 83, wherein the packaging is for a food product.

85. A bacteria detecting composition comprising a diatom and a sensor.

86. The bacteria detecting composition of clause 85, wherein the diatom is a diatom frustule.

87. The bacteria detecting composition of clause 85, wherein the diatom is a diatomite.

88. The bacteria detecting composition of clause 85, wherein the diatom is diatomaceous earth.

89. The bacteria detecting composition of any one of clauses 85 to 88, wherein the surface of the diatom comprises one or more pores.

90. The bacteria detecting composition of clause 89, wherein at least part of the sensor is contained within one or more pores in the diatom.

91. The bacteria detecting composition of any one of clauses 85 to 90, wherein the sensor is an organic reagent.

92. The bacteria detecting composition of any one of clauses 85 to 90, wherein the sensor is an inorganic reagent.

93. The bacteria detecting composition of any one of clauses 85 to 90, wherein the sensor is a dye.

94. The bacteria detecting composition of any one of clauses 85 to 90, wherein the sensor is an enzyme.

95. The bacteria detecting composition of any one of clauses 85 to 90, wherein the sensor is a salt.

96. The bacteria detecting composition of clause 95, wherein the salt is an XG salt.

97. The bacteria detecting composition of any one of clauses 85 to 90, wherein the sensor is a fluorescent agent.

98. The bacteria detecting composition of any one of clauses 85 to 90, wherein the sensor is DNA.

99. The bacteria detecting composition of any one of clauses 85 to 90, wherein the sensor is a protein.

100. The bacteria detecting composition of any one of clauses 85 to 90, wherein the sensor is an antibody.

101. The bacteria detecting composition of any one of clauses 85 to 90, wherein the sensor is a quantum dot.

102. The bacteria detecting composition of any one of clauses 85 to 101, wherein the sensor changes color following exposure to bacteria.

103. The bacteria detecting composition of clause 102, wherein the bacteria is an airborne bacteria.

104. The bacteria detecting composition of any one of clauses 85 to 101, wherein the sensor changes color following exposure to an enzyme.

105. A bacteria detecting system comprising the bacteria detecting composition of clause 85 and an article.

106. The bacteria detecting system of clause 105, wherein the bacteria detecting composition is applied to the article.

107. The bacteria detecting system of clause 106, wherein the bacteria detecting composition is applied to the article using a polymer binder.

108. The bacteria detecting system of any one of clauses 105 to 107, wherein the article is paper.

109. The bacteria detecting system of clause 108, wherein the bacteria detecting composition is applied to the paper using inkjet printing.

110. The bacteria detecting system of any one of clauses 105 to 107, wherein the article is a tissue towel.

111. The bacteria detecting system of any one of clauses 105 to 107, wherein the article is a nonwoven.

112. The bacteria detecting system of any one of clauses 105 to 107, wherein the article is a gas permeable coating.

113. The bacteria detecting system of any one of clauses 105 to 107, wherein the article is a gas permeable paint.

114. The bacteria detecting system of any one of clauses 105 to 107, wherein the article is a storage wall.

115. The bacteria detecting system of any one of clauses 105 to 107, wherein the article is a filter.

116. The bacteria detecting system of any one of clauses 105 to 107, wherein the article is a felt tip.

117. An explosive degradation product detecting composition comprising a diatom and a sensor.

118. The explosive degradation product detecting composition of clause 117, wherein the diatom is a diatom frustule.

119. The explosive degradation product detecting composition of clause 117, wherein the diatom is a diatomite.

120. The explosive degradation product detecting composition of clause 117, wherein the diatom is diatomaceous earth.

121. The explosive degradation product detecting composition of any one of clauses 117 to 120, wherein the surface of the diatom comprises one or more pores.

122. The explosive degradation product detecting composition of clause 121, wherein at least part of the sensor is contained within one or more pores in the diatom.

123. The explosive degradation product detecting composition of any one of clauses 117 to 122, wherein the sensor is an organic reagent.

124. The explosive degradation product detecting composition of any one of clauses 117 to 122, wherein the sensor is an inorganic reagent.

125. The explosive degradation product detecting composition of any one of clauses 117 to 122, wherein the sensor is a dye.

126. The explosive degradation product detecting composition of any one of clauses 117 to 122, wherein the sensor is an enzyme.

127. The explosive degradation product detecting composition of any one of clauses 117 to 122, wherein the sensor is a salt.

128. The explosive degradation product detecting composition of clause 127, wherein the salt is an XG salt.

129. The explosive degradation product detecting composition of any one of clauses 117 to 122, wherein the sensor is a fluorescent agent.

130. The explosive degradation product detecting composition of any one of clauses 117 to 122, wherein the sensor is DNA.

131. The explosive degradation product detecting composition of any one of clauses 117 to 122, wherein the sensor is a protein.

132. The explosive degradation product detecting composition of any one of clauses 117 to 122, wherein the sensor is an antibody.

133. The explosive degradation product detecting composition of any one of clauses 117 to 122, wherein the sensor is a quantum dot.

134. The explosive degradation product detecting composition of any one of clauses 117 to 133, wherein the sensor changes color following exposure to an explosive degradation product.

135. The explosive degradation product detecting composition of clause 134, wherein the explosive degradation product is nitrogen.

136. The explosive degradation product detecting composition of clause 134, wherein the explosive degradation product is ammonia.

137. A explosive degradation product detecting system comprising the explosive degradation product detecting composition of clause 301 and an article.

138. The explosive degradation product detecting system of clause 137, wherein the explosive degradation product detecting composition is applied to the article.

139. The explosive degradation product detecting system of clause 138, wherein the explosive degradation product detecting composition is applied to the article using a polymer binder.

140. The explosive degradation product detecting system of any one of clauses 137 to 139, wherein the article is paper.

141. The explosive degradation product detecting system of clause 140, wherein the explosive degradation product detecting composition is applied to the paper using inkjet printing.

142. The explosive degradation product detecting system of any one of clauses 137 to 139, wherein the article is a tissue towel.

143. The explosive degradation product detecting system of any one of clauses 137 to 139, wherein the article is a nonwoven.

144. The explosive degradation product detecting system of any one of clauses 137 to 139, wherein the article is a gas permeable coating.

145. The explosive degradation product detecting system of any one of clauses 137 to 139, wherein the article is a gas permeable paint.

146. The explosive degradation product detecting system of any one of clauses 137 to 139, wherein the article is a storage wall.

147. The explosive degradation product detecting system of any one of clauses 137 to 139, wherein the article is a filter.

148. The explosive degradation product detecting system of any one of clauses 137 to 139, wherein the article is a felt tip.

149. A neurotoxin detecting composition comprising a diatom and a sensor.

150. The neurotoxin detecting composition of clause 149, wherein the diatom is a diatom frustule.

151. The neurotoxin detecting composition of clause 149, wherein the diatom is a diatomite.

152. The neurotoxin detecting composition of clause 149, wherein the diatom is diatomaceous earth.

153. The neurotoxin detecting composition of any one of clauses 149 to 152, wherein the surface of the diatom comprises one or more pores.

154. The neurotoxin detecting composition of clause 153, wherein at least part of the sensor is contained within one or more pores in the diatom.

155. The neurotoxin detecting composition of any one of clauses 149 to 154, wherein the sensor is an organic reagent.

156. The neurotoxin detecting composition of any one of clauses 149 to 154, wherein the sensor is an inorganic reagent.

157. The neurotoxin detecting composition of any one of clauses 149 to 156, wherein the sensor comprises DTNB and AtCh.

158. The neurotoxin detecting composition of any one of clauses 149 to 157, wherein the sensor changes color following exposure to a neurotoxin.

159. The neurotoxin detecting composition of any one of clauses 149 to 157, wherein the sensor does not change color following exposure to a neurotoxin.

160. The neurotoxin detecting composition of clause 158 or clause 159, wherein the neurotoxin is an acetylcholinesterase inhibitor.

161. A neurotoxin detecting system comprising the neurotoxin detecting composition of clause 149 and an article.

162. The neurotoxin detecting system of clause 161, wherein the neurotoxin detecting composition is applied to the article.

163. The neurotoxin detecting system of clause 162, wherein the neurotoxin detecting composition is applied to the article using a polymer binder.

164. The neurotoxin detecting system of any one of clauses 161 to 163, wherein the article is paper.

165. The neurotoxin detecting system of clause 164, wherein the neurotoxin detecting composition is applied to the paper using inkjet printing.

166. The neurotoxin detecting system of any one of clauses 161 to 163, wherein the article is a tissue towel.

167. The neurotoxin detecting system of any one of clauses 161 to 163, wherein the article is a nonwoven.

168. The neurotoxin detecting system of any one of clauses 161 to 163, wherein the article is a gas permeable coating.

169. The neurotoxin detecting system of any one of clauses 161 to 163, wherein the article is a gas permeable paint.

170. The neurotoxin detecting system of any one of clauses 161 to 163, wherein the article is a storage wall.

171. The neurotoxin detecting system of any one of clauses 161 to 163, wherein the article is a filter.

172. The neurotoxin detecting system of any one of clauses 161 to 163, wherein the article comprises a felt tip.

173. The neurotoxin detecting system of any one of clauses 161 to 163, wherein the article comprises a felt tip and a pen-like device.

174. The neurotoxin detecting system of any one of clauses 161 to 163 or clause 173, wherein the article comprises an ampoule comprising acetylcholinesterase.

175. The neurotoxin detecting system of clause 174, wherein the acetylcholinesterase is a lyophilized enzyme and the ampoule further comprises water.

176. A method of identifying the presence of a gas associated with a food product, said method comprising the steps of

a) applying a gas detection composition to an article, wherein the gas detection composition comprises a diatom and a sensor;

b) placing the article near the food product for a period of time;

c) identifying a change in color of the sensor; and

d) identifying the presence of the gas associated with the food product, wherein the change in color of the sensor indicates the presence of the gas associated with the food product.

177. The method of clause 176, wherein the diatom is a diatom frustule.

178. The method of clause 176, wherein the diatom is a diatomite.

179. The method of clause 176, wherein the diatom is diatomaceous earth.

180. The method of any one of clauses 176 to 179, wherein the surface of the diatom comprises one or more pores.

181. The method of clause 180, wherein at least part of the sensor is contained within one or more pores in the diatom.

182. The method of any one of clauses 176 to 181, wherein the sensor is an organic reagent.

183. The method of any one of clauses 176 to 181, wherein the sensor is an inorganic reagent.

184. The method of any one of clauses 176 to 181, wherein the sensor is a dye.

185. The method of any one of clauses 176 to 181, wherein the sensor is an enzyme.

186. The method of any one of clauses 176 to 181, wherein the sensor is a salt.

187. The method of clause 186, wherein the salt is an XG salt.

188. The method of any one of clauses 176 to 181, wherein the sensor is a fluorescent agent.

189. The method of any one of clauses 176 to 181, wherein the sensor is DNA.

190. The method of any one of clauses 176 to 181, wherein the sensor is a protein.

191. The method of any one of clauses 176 to 181, wherein the sensor is an antibody.

192. The method of any one of clauses 176 to 181, wherein the sensor is a quantum dot.

193. The method of any one of clauses 176 to 192, wherein the gas is a nitrogen-containing gas.

194. The method of any one of clauses 176 to 193, wherein the gas is a total volatile basic nitrogen (TVB-N) gas.

195. The method of any one of clauses 176 to 194, wherein the gas is ammonia.

196. The method of any one of clauses 176 to 195, wherein the sensor changes color following exposure to volatile basic nitrogen.

197. The method of any one of clauses 176 to 196, wherein the sensor changes color following a change in pH.

198. The method of any one of clauses 176 to 196, wherein the sensor changes color following a change in temperature.

199. The method of any one of clauses 176 to 198, wherein the gas detecting composition is applied to the article using a polymer binder.

200. The method of any one of clauses 176 to 199, wherein the article is paper.

201. The method of clause 200, wherein the gas detecting composition is applied to the paper using inkjet printing.

202. The method of clause 200 or clause 201, wherein the paper is a sticker.

203. The method of clause 200 or clause 201, wherein the paper is a label.

204. The method of any one of clauses 200 to 203, wherein the paper comprises packaging for a food product.

205. The method of any one of clauses 176 to 203, wherein the article is packaging.

206. The method of clause 205, wherein the diatom is applied to the packaging using inkjet printing.

207. The method of clause 205 or 206, wherein the packaging is for a food product.

208. The method of any one of clauses 176 to 207, wherein the identification of the presence of the gas associated with the food product has a sensitivity of about 5 parts per million.

209. A method of detecting a bacteria associated with a product, said method comprising the steps of

a) applying a bacteria detection composition to an article, wherein the bacteria detection composition comprises a diatom and a sensor;

b) placing the article near the product for a period of time;

c) identifying a change in color of the sensor; and

d) identifying the presence of the bacteria associated with the product, wherein the change in color of the sensor indicates the presence of the bacteria associated with the product.

210. The method of clause 209, wherein the diatom is a diatom frustule.

211. The method of clause 209, wherein the diatom is a diatomite.

212. The method of clause 209, wherein the diatom is diatomaceous earth.

213. The method of any one of clauses 209 to 212, wherein the surface of the diatom comprises one or more pores.

214. The method of clause 209, wherein at least part of the sensor is contained within one or more pores in the diatom.

215. The method of any one of clauses 209 to 214, wherein the sensor is an organic reagent.

216. The method of any one of clauses 209 to 214, wherein the sensor is an inorganic reagent.

217. The method of any one of clauses 209 to 214, wherein the sensor is a dye.

218. The method of any one of clauses 209 to 214, wherein the sensor is an enzyme.

219. The method of any one of clauses 209 to 214, wherein the sensor is a salt.

220. The method of clause 219, wherein the salt is an XG salt.

221. The method of any one of clauses 209 to 214, wherein the sensor is a fluorescent agent.

222. The method of any one of clauses 209 to 214, wherein the sensor is DNA.

223. The method of any one of clauses 209 to 214, wherein the sensor is a protein.

224. The method of any one of clauses 209 to 214, wherein the sensor is an antibody.

225. The method of any one of clauses 209 to 214, wherein the sensor is a quantum dot.

226. The method of any one of clauses 209 to 225, wherein the bacteria is an airborne bacteria.

227. The method of any one of clauses 209 to 226, wherein the bacteria detecting composition is applied to the article using a polymer binder.

228. The method of any one of clauses 209 to 227, wherein the article is paper.

229. The method of clause 228, wherein the bacteria detecting composition is applied to the paper using inkjet printing.

230. The method of any one of clauses 209 to 227, wherein the article is a tissue towel.

231. The method of any one of clauses 209 to 227, wherein the article is a nonwoven.

232. The method of any one of clauses 209 to 227, wherein the article is a gas permeable coating.

233. The method of any one of clauses 209 to 227, wherein the article is a gas permeable paint.

234. The method of any one of clauses 209 to 227, wherein the article is a storage wall.

235. The method of any one of clauses 209 to 227, wherein the article is a filter.

236. The method of any one of clauses 209 to 227, wherein the article is a felt tip.

237. A method of detecting an explosive degradation product, said method comprising the steps of

a) applying a explosive degradation product detection composition to an article, wherein the explosive degradation product detection composition comprises a diatom and a sensor;

b) placing the article near a suspected explosive device for a period of time;

c) identifying a change in color of the sensor; and

d) identifying the presence of the explosive degradation product associated with the suspected explosive device, wherein the change in color of the sensor indicates the presence of the explosive degradation product associated with the suspected explosive device.

238. The method of clause 237, wherein the diatom is a diatom frustule.

239. The method of clause 237, wherein the diatom is a diatomite.

240. The method of clause 237, wherein the diatom is diatomaceous earth.

241. The method of any one of clauses 237 to 240, wherein the surface of the diatom comprises one or more pores.

242. The method of clause 241, wherein at least part of the sensor is contained within one or more pores in the diatom.

243. The method of any one of clauses 237 to 242, wherein the sensor is an organic reagent.

244. The method of any one of clauses 237 to 242, wherein the sensor is an inorganic reagent.

245. The method of any one of clauses 237 to 242, wherein the sensor is a dye.

246. The method of any one of clauses 237 to 242, wherein the sensor is an enzyme.

247. The method of any one of clauses 237 to 242, wherein the sensor is a salt.

248. The method of clause 247, wherein the salt is an XG salt.

249. The method of any one of clauses 237 to 242, wherein the sensor is a fluorescent agent.

250. The method of any one of clauses 237 to 242, wherein the sensor is DNA.

251. The method of any one of clauses 237 to 242, wherein the sensor is a protein.

252. The method of any one of clauses 237 to 242, wherein the sensor is an antibody.

253. The method of any one of clauses 237 to 242, wherein the sensor is a quantum dot.

254. The method of any one of clauses 237 to 253, wherein the sensor changes color following exposure to an explosive degradation product.

255. The method of any one of clauses 237 to 254, wherein the explosive degradation product is nitrogen.

256. The method of any one of clauses 237 to 254, wherein the explosive degradation product is ammonia.

257. The method of any one of clauses 237 to 256, wherein the explosive degradation product detecting composition is applied to the article using a polymer binder.

258. The method of any one of clauses 237 to 257, wherein the article is paper.

259. The method of clause 258, wherein the explosive degradation product detecting composition is applied to the paper using inkjet printing.

260. The method of any one of clauses 237 to 257, wherein the article is a tissue towel.

261. The method of any one of clauses 237 to 257, wherein the article is a nonwoven.

262. The method of any one of clauses 237 to 257, wherein the article is a gas permeable coating.

263. The method of any one of clauses 237 to 257, wherein the article is a gas permeable paint.

264. The method of any one of clauses 237 to 257, wherein the article is a storage wall.

265. The method of any one of clauses 237 to 257, wherein the article is a filter.

266. The method of any one of clauses 237 to 257, wherein the article is a felt tip.

267. A method of detecting a neurotoxin on a product, said method comprising the steps of

a) applying a neurotoxin detection composition to an article, wherein the neurotoxin detection composition comprises a diatom and a sensor;

b) contacting the article and the product;

c) identifying a change in color of the sensor; and

d) identifying the presence of the neurotoxin on the product, wherein a lack of change in color of the sensor indicates the presence of the neurotoxin on the product.

268. The method of clause 267, wherein the diatom is a diatom frustule.

269. The method of clause 267, wherein the diatom is a diatomite.

270. The method of clause 267, wherein the diatom is diatomaceous earth.

271. The method of any one of clauses 267 to 270, wherein the surface of the diatom comprises one or more pores.

272. The method of clause 271, wherein at least part of the sensor is contained within one or more pores in the diatom.

273. The method of any one of clauses 267 to 272, wherein the sensor is an organic reagent.

274. The method of any one of clauses 267 to 272, wherein the sensor is an inorganic reagent.

275. The method of any one of clauses 267 to 272, wherein the sensor comprises DTNB and AtCh.

276. The method of any one of clauses 267 to 275, wherein the neurotoxin is an acetylcholinesterase inhibitor.

277. The method of any one of clauses 267 to 276, wherein the neurotoxin detecting composition is applied to the article using a polymer binder.

278. The method of any one of clauses 267 to 277, wherein the article is paper.

279. The method of clause 278, wherein the neurotoxin detecting composition is applied to the paper using inkjet printing.

280. The method of any one of clauses 267 to 277, wherein the article is a tissue towel.

281. The method of any one of clauses 267 to 277, wherein the article is a nonwoven.

282. The method of any one of clauses 267 to 277, wherein the article is a gas permeable coating.

283. The method of any one of clauses 267 to 277, wherein the article is a gas permeable paint.

284. The method of any one of clauses 267 to 277, wherein the article is a storage wall.

285. The method of any one of clauses 267 to 277, wherein the article is a filter.

286. The method of any one of clauses 267 to 277, wherein the article comprises a felt tip.

287. The method of any one of clauses 267 to 277, wherein the article comprises a felt tip and a pen-like device.

288. The method of clause 287, wherein the article further comprises an ampoule comprising acetylcholinesterase.

289. The method of clause 288, wherein the acetylcholinesterase is a lyophilized enzyme and the ampoule further comprises water.

### BRIEF DESCRIPTION OF THE DRAWINGS

FIG. 1 shows an SEM picture demonstrating diatoms applied to the surface of paper (magnification 4.00 K×).

FIG. 2 shows an SEM picture demonstrating diatoms applied to the surface of paper (magnification 1.00 K×).

FIG. 3 shows four different coating formulations applied to the paper-based diatom sensors in the various tubes: (a) the coating formulation applied at 6 g/m² coat weight and containing 0.5 parts of dye; (b) the coating formulation applied at 20 g/m² coat weight and containing 0.5 parts of dye; (c) the coating formulation applied at 6 g/m² coat weight and containing 1 part of dye; (d) the coating formulation applied at 20 g/m² coat weight and containing 1 part of dye.

FIG. 4 shows a correlation of total bacterial colony forming units (CFU) per gram of fillet over a 13 day period for catfish and tilapia.

FIG. 5 shows the mechanism of action for detecting bacteria (e.g., E. coli) using the diatom sensor compositions.

FIG. 6 shows detection of acetylcholinesterase (AChE) enzyme detected using the diatom sensor compositions. The top panel shows detection of AChE on ceramic tile. The bottom panel shows AChE enzyme immobilized on paper with no ATCh or DTNB incorporated into the diatoms (Box (a)) and AChE enzyme immobilized on paper with ATCh and DTNB incorporated into the diatoms (Box (b)).

FIG. 7 shows the mechanism of action for detecting neurotoxin (e.g., AChE inhibitors) using the diatom sensor compositions.

Various embodiments of the invention are described herein as follows. In one embodiment described herein, a composition comprising a diatom and a sensor is provided. In another embodiment, a gas detecting composition comprising a diatom and a sensor is provided. In yet another embodiment, a gas detecting system is provided. The gas detecting system comprises the gas detecting composition, comprising a diatom and a sensor, and an article.

In another embodiment, a bacteria detecting composition comprising a diatom and a sensor is provided. In yet another embodiment, a bacteria detecting system is provided. The bacteria detecting system comprises the bacteria detecting composition, comprising a diatom and a sensor, and an article.

In another embodiment, an explosive degradation product detecting composition comprising a diatom and a sensor is provided. In yet another embodiment, an explosive degradation product detecting system is provided. The explosive degradation product detecting system comprises the explosive degradation product detecting composition, comprising a diatom and a sensor, and an article.

In another embodiment, a neurotoxin detecting composition comprising a diatom and a sensor is provided. In yet another embodiment, a neurotoxin detecting system is provided. The neurotoxin detecting system comprises the neurotoxin detecting composition, comprising a diatom and a sensor, and an article.

In another embodiment, a method of identifying the presence of a gas associated with a food product is provided. The method comprises the steps of a) applying a gas detection composition to an article, wherein the gas detection composition comprises a diatom and a sensor; b) placing the article near the food product for a period of time; c) identifying a change in color of the sensor; and d) identifying the presence of the gas associated with the food product, wherein the change in color of the sensor indicates the presence of the gas associated with the food product.

In another embodiment, a method of detecting a bacteria associated with a product is provided. The method comprises the steps of a) applying a bacteria detection composition to an article, wherein the bacteria detection composition comprises a diatom and a sensor; b) placing the article near the product for a period of time; c) identifying a change in color of the sensor; and d) identifying the presence of the bacteria associated with the product, wherein the change in color of the sensor indicates the presence of the bacteria associated with the product.

In another embodiment, a method of detecting an explosive degradation product is provided. The method comprises the steps of a) applying a explosive degradation product detection composition to an article, wherein the explosive degradation product detection composition comprises a diatom and a sensor; b) placing the article near a suspected explosive device for a period of time; c) identifying a change in color of the sensor; and d) identifying the presence of the explosive degradation product associated with the suspected explosive device, wherein the change in color of the sensor indicates the presence of the explosive degradation product associated with the suspected explosive device.

In another embodiment, a method of detecting a neurotoxin on a product is provided. The method comprises the steps of a) applying a neurotoxin detection composition to an article, wherein the neurotoxin detection composition comprises a diatom and a sensor; b) contacting the article and the product; c) identifying a change in color of the sensor; and d) identifying the presence of the neurotoxin on the product, wherein a lack of change in color of the sensor indicates the presence of the neurotoxin on the product.

In the various embodiments, the composition comprises a diatom and a sensor. The term “diatom” refers to unicellular algae that are encased within a silica cell wall. Diatoms include both centric diatoms and pinnate diatoms, and are one of the most common types of phytoplankton.

Diatoms comprise a self-assembled micro- and nano-porous silica outer cell wall called a “frustule,” in which the diatom cells are contained. A frustule comprises hydrated glass (SiO₂.nH₂O) with naturally formed micro- and nano-fabricated two-dimensional pore arrays.

Following algal death, the remnant frustule is referred to as “diatomite” or “diatomaceous earth.” As used herein, the term “diatom” refers to a diatom frustule, a diatomite, or a diatomaceous earth. These terms are well known in the art of algae. Importantly, diatoms are natural, self-assembled nanostructures that are abundantly available.

In some embodiments, the surface of the diatom comprises one or more pores. As used herein, the term “pore” refers to a cavity in the diatom. Diatoms typically have thousands of pores on their surface, and can be of several distinct sizes. The pores in the surface of the diatom can have a diameter between about 0.01 nm and about 100 nm, between about 0.1 nm and about 50 nm, between about 1 nm and about 10 nm, and the like. This morphologic feature of the diatom can present the structure as a tube with both micropores and mesopores. Importantly, the structure allows for a high surface area, for example 30.0 m² per gram or more.

In addition, the pores on the surface of the diatom can create vessels in the diatom that are capable of maximizing the flow of gas, liquid, and other materials through the diatom. Diatoms are available in a number of various shapes and quantity of pores. Furthermore, some species of diatoms have formed vessels that can influence the gas permeability of the diatoms, as well as the absorption of gases, liquids, salts and enzymes by the structures.

As used herein, the term “sensor” refers a substance that is responsive to chemical analytes and/or to environmental stimuli. In some embodiments, at least part of the sensor is contained within one or more pores in the diatom. For example, the sensor may be incorporated inside one or more pores in the diatom, depending on the procedure in which the sensor is applied to the diatom.

In some embodiments, the sensor is an organic reagent. In other embodiments, the sensor is an inorganic reagent. In yet other embodiments, the sensor is a dye. As used herein, the term “dye” refers to an aromatic molecule capable of absorbing light in the spectral range of from about 250 nm to about 1,200 nm, inclusive. Generally, the term “dye” may refer to a fluorescent dye, a non-fluorescent dye, or both. For example, dyes can include pH sensitive dyes. Many pH sensitive dyes are known in the art and may be used with the present disclosure, for example bromocresol purple or bromocresol green.

In other embodiments, the sensor is an enzyme. As used herein, the term “enzyme” has the general meaning in the art and refers any substance composed wholly or largely of protein or polypeptides that catalyzes or promotes, more or less specifically, one or more chemical or biochemical reactions.

In other embodiments, the sensor is a salt. As used herein, the term “salt” has the general meaning in the art and refers to any ionic form of a compound and one or more counter-ionic species (cations and/or anions). Salts also include zwitterionic compounds (i.e., a molecule containing one more cationic and anionic species, e.g., zwitterionic amino acids). Counter ions present in a salt can include any cationic, anionic, or zwitterionic species. Exemplary anions include, but are not limited to: chloride, bromide, iodide, nitrate, sulfate, bisulfate, sulfite, bisulfite, phosphate, acid phosphate, perchl orate, chlorate, chlorite, hypochlorite, periodate, iodate, iodite, hypoiodite, carbonate, bicarbonate, isonicotinate, acetate, trichloroacetate, trifluoroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, trifluormethansulfonate, ethanesulfonate, benzensulfonate, p-toluenesulfonate, p-trifluoromethylbenzenesulfonate, hydroxide, aluminates and borates. Exemplary cations include, but are not limited to: monovalent alkali metal cations, such as lithium, sodium, potassium, and cesium, and divalent alkaline earth metals, such as beryllium, magnesium, calcium, strontium, and barium. Also included are transition metal cations, such as gold, silver, copper and zinc, as well as non-metal cations, such as ammonium salts. In one embodiment, the salt is an XG salt (i.e., 5-bromo-4-chloro-3-indolyl-β-D-glusormide-sodium salts).

In other embodiments, the sensor is a fluorescent agent. As used herein, the term “fluorescent agent” refers to an agent that provides a fluorescence signal. In yet other embodiments, the sensor is DNA. In some embodiments, the sensor is a protein. In other embodiments, the sensor is an antibody. In yet other embodiments, the sensor is a quantum dot.

The terms “DNA,” “protein,” “antibody,” and “quantum dot” are well known and refer to their general meanings in the art.

In some embodiments, the sensor changes color following exposure to a gas. A change in color can refer to any change in visible color of the sensor. Furthermore, a change in color can refer to a change in color of the sensor that is detectable by other measures known in the art, such as color detecting instrumentation. For example, in some embodiments, the sensor may turn blue or turn yellow.

As used herein, the term “gas” refers to its general meaning in the art. In some embodiments, the gas is a nitrogen-containing gas. In other embodiments, the gas is a total volatile basic nitrogen (TVB-N) gas. As used herein, the term TVB-N refers to the amount of basic, nitrogen-containing chemicals distilled from an alkalized extract or suspension of a food product. For example, TVB-N can be analyzed from any type of meat product, such as beef, pork, poultry, fisheries, and the like. The bases, amines, in the distillate are determined by titration with standard acid. The bases (amines) have one basic nitrogen atom in the molecule and TVB-N is expressed on a nitrogen basis (typically milligrams of nitrogen/100 grams of sample. Typically, the main component of TVB-N is ammonia (NH₃). In some embodiments, the gas is ammonia.

In some embodiments, the sensor changes color following a change in pH. Various pH sensitive dyes are known in the art, for example, bromocresol purple and bromocresol green. In other embodiments, the sensor changes color following a change in temperature. In yet other embodiments, the sensor changes color following exposure to a neurotoxin. As used herein, the term “neurotoxin” refers to a substance or toxin that causes damage to a nerve or nerve tissue, for example, an acetylcholinesterase inhibitor, paraoxon, a snake venom, aflatoxin B, nerve gas, Sarin, VX, OP compounds, and the like. In one embodiment, the neurotoxin is an acetylcholinesterase inhibitor. As used herein the term “acetylcholinesterase inhibitor” refers to any compound or substance that inhibits the enzyme acetylcholinesterase.

In other embodiments, the sensor changes color following exposure to bacteria. In some embodiments, the bacteria is an airborne bacteria. As used herein, the term “airborne bacteria” refers to a bacteria transmitted through the air.

In other embodiments, the sensor changes color following exposure to an enzyme. In yet other embodiments, the sensor changes color following exposure to an explosive degradation product. As used herein, the term “explosive degradation product” refers to any product formed via degradation of an explosive component. In one embodiment, the explosive degradation product is nitrogen. In another embodiment, the explosive degradation product is ammonia.

In various embodiments, the composition further comprises an article, wherein the diatom is applied to the article. The term “article” encompasses any physical object to which the diatom may be applied. As used herein, the term “applied” refers to any appropriate method of affixing the diatom to the article, such as incorporating, imbedding, impregnating, encapsulating, embedding, immobilizing, and the like. In some embodiments, the diatom is applied to the article using a polymer binder. The term “polymer binder” has a general meaning in the art, and includes starches, proteins, latexes, caseins, carboxy methyl cellulose, nano/micro-fibrillated cellulose, and the like, and can be used alone or in combination. For example, polyvinyl alcohol (PVOH) and latex binders such as high glass transition temperature latex binders can be utilized as polymer binders.

The application of the composition to an article can also include one or more functional additives to assist in the application. For example, cationic additives such as Poly-DADMAC, polyvinylamine, and low molecular weight cationic polymers (e.g., polyethylenimine) can be used to enhance the application of the diatom to the article. Other additives include glyoxal modified polyacrylamide or polyamide epichlorohydrine. Furthermore, ion pairing with a quaternary ammonium salt such as cetyl trimethyl ammonium, bromide, octadecyl ammonium bromide, tetrahexyl ammonium bromide, and/or tetraoctyl ammonium bromide can be utilized.

Alternatively, application techniques can be based on adsorption, absorption, electrostatic charge, and covalent attachment, covalent bonding, or physical encapsulation.

In some embodiments, the article is paper. In other embodiments, the diatom is applied to the paper using inkjet printing. Inkjet printing is well known in the art and includes applications that are adaptable to existing printing methods. Inkjet printing offers an advantageous option for applying the compositions of the present disclosure at point-of-use directly on articles. Sensors can be applied to paper using an inkjet printer (e.g., Dimatix Materials Printer DMP-2800; Fujifilm). Inkjet ink involved different rheological properties than coating formulations, for example differing viscosity and surface tension. Rheology using these two parameters can be adjusted by varying solids content, use of glycerin (viscosity), and use of surfactants (surface tension). In one embodiment, the paper is a sticker. In another embodiment, the paper is a label.

In yet other embodiments, the paper comprises packaging for a food product. The term “packaging” as used herein refers to any containment of food products, both in packaging intended for distribution of the food products to consumers and packaging intended for manufacturing, storing and other steps in the production of food or beverage products. The term “food product” as used herein refers to any food that is susceptible to spoilage as a result of bacterial growth and proliferation on the surface of the food. Such food products include, but are not limited to meat, vegetables, fruits and grains.

As used herein, the term “meat” refers to any fresh meat product or meat by-product from an animal of the kingdom Animalia which is consumed by humans or animals, including without limitation meat from bovine, ovine, porcine, poultry, fish and crustaceous seafood. It is contemplated that the present invention can be applied to the preservation of non-animal food products, such as fruits, vegetables and grains, subject to spoilage by bacterial microorganisms. Such bacterial microorganisms can be referred to as “spoilage bacteria” and include Escherichia coli (E. coli), Salmonella species, and any other bacteria known to cause spoilage of food products.

Spoilage bacteria may grow and proliferate to such a degree that a food product is made unsuitable or undesirable for human or animal consumption. Bacteria are able to proliferate on food surfaces, such as meat surfaces, by assimilating sugars and proteins on such surfaces, resulting in microbial outgrow. By metabolizing these components, spoilage bacteria create by-products including carbon dioxide, methane, nitrogenous compounds, butyric acid, propionic acid, lactic acid, formic acid, sulfur compounds, and other undesired gases and acids. The production of such by-products alters the color of meat surfaces, often turning meat from a red color to a brown, grey, or green color. Gaseous by-products generated by spoilage bacteria also give spoiled meat an undesirable odor. The color and odor alterations of meat due to the growth of spoilage bacteria on a meat product surface often make such meat unsalable to consumers.

In some embodiments, the article is packaging. In other embodiments, the diatom is applied to the packaging using inkjet printing. In yet other embodiments, the packaging is for a food product.

In some embodiments, the article is a tissue towel. In other embodiments, the article is a nonwoven. As used herein, the term “nonwoven” refers herein to a material made from continuous (long) filaments (fibers) and/or discontinuous (short) filaments (fibers) by processes such as spunbonding, meltblowing, and the like.

In some embodiments, the article is a gas permeable coating. In other embodiments, the article is a gas permeable paint. The term “gas permeable” has its general meaning in the art. In yet other embodiments, the article is a storage wall. In some embodiments, the article is a filter. In other embodiments, the article is a felt tip.

In another aspect of the present disclosure, a gas detecting composition is provided. The gas detecting composition comprises a diatom and a sensor. The previously described embodiments of the composition comprising a diatom and a sensor are applicable to the gas detecting composition described herein.

In another aspect of the present disclosure, a gas detecting system is provided. The gas detecting system comprises the gas detecting composition (i.e., comprising a diatom and a sensor) and an article. The previously described embodiments of the composition comprising a diatom and a sensor are applicable to the gas detecting system described herein.

In another aspect of the present disclosure, a gas detecting composition is provided. The gas detecting composition comprises a diatom and a sensor. The previously described embodiments of the composition comprising a diatom and a sensor are applicable to the gas detecting composition described herein.

In another aspect of the present disclosure, a gas detecting system is provided. The gas detecting system comprises the gas detecting composition (i.e., comprising a diatom and a sensor) and an article. The previously described embodiments of the composition comprising a diatom and a sensor are applicable to the gas detecting system described herein.

In another aspect of the present disclosure, a bacteria detecting composition is provided. The bacteria detecting composition comprises a diatom and a sensor. The previously described embodiments of the composition comprising a diatom and a sensor are applicable to the bacteria detecting composition described herein.

In another aspect of the present disclosure, a bacteria detecting system is provided. The bacteria detecting system comprises the gas detecting composition (i.e., comprising a diatom and a sensor) and an article. The previously described embodiments of the composition comprising a diatom and a sensor are applicable to the bacteria detecting system described herein.

In another aspect of the present disclosure, an explosive degradation product detecting composition is provided. The explosive degradation product detecting composition comprises a diatom and a sensor. The previously described embodiments of the composition comprising a diatom and a sensor are applicable to the explosive degradation product detecting composition described herein.

In another aspect of the present disclosure, an explosive degradation product detecting system is provided. The explosive degradation product detecting system comprises the explosive degradation product detecting composition (i.e., comprising a diatom and a sensor) and an article. The previously described embodiments of the composition comprising a diatom and a sensor are applicable to the explosive degradation product detecting system described herein.

In another aspect of the present disclosure, a neurotoxin detecting composition is provided. The neurotoxin detecting composition comprises a diatom and a sensor. The previously described embodiments of the composition comprising a diatom and a sensor are applicable to the neurotoxin detecting composition described herein. In some embodiments, the sensor of the neurotoxin detecting composition comprises dithiobisnitrobenzoate (DTNB) and acetylthiocholine (ATCh). In one embodiment, the sensor of the neurotoxin detecting composition changes color following exposure to a neurotoxin. In another embodiment, the sensor of the neurotoxin detecting composition does not change color following exposure to a neurotoxin.

In another aspect of the present disclosure, a neurotoxin detecting system is provided. The neurotoxin detecting system comprises the neurotoxin detecting composition (i.e., comprising a diatom and a sensor) and an article. The previously described embodiments of the composition comprising a diatom and a sensor are applicable to the neurotoxin detecting system described herein. In some embodiments, the article comprises a felt tip and a pen-like device. In another embodiment, the article comprises an ampoule comprising acetylcholinesterase. In yet another embodiment, the acetylcholinesterase is a lyophilized enzyme and the ampoule further comprises water. The term “lyophilization,” also known as freeze-drying, refers to the technique to remove water from the preparation of interest. Lyophilization is a process by which the material to be dried is first frozen and then the ice or frozen solvent is removed by sublimation in a vacuum environment.

In another aspect of the present disclosure, a method of identifying the presence of a gas associated with a food product is provided. The method comprises the steps of a) applying a gas detection composition to an article, wherein the gas detection composition comprises a diatom and a sensor; b) placing the article near the food product for a period of time; c) identifying a change in color of the sensor; and d) identifying the presence of the gas associated with the food product, wherein the change in color of the sensor indicates the presence of the gas associated with the food product. The previously described embodiments of the composition comprising a diatom and a sensor, of the gas detecting composition, and of the gas detecting system are applicable to the method of identifying the presence of a gas associated with a food product described herein.

The method of identifying the presence of a gas associated with a food product includes the step of placing the article near the food product for a period of time. As used herein, the term “placing” means positioning the article in the vicinity of the food product, for example in or on the packaging of the food product. For example, placing the article near the food product includes placing the article in the headspace of the food product (i.e., the unfilled space left between the food and the packaging). Any suitable period of time can be utilized according to the present invention to identify the presence of the gas (e.g., TVB-N basis). In one embodiment, the identification of the presence of the gas associated with the food product has a sensitivity of about 5 parts per million.

In another aspect of the present disclosure, a method of detecting a bacteria associated with a product is provided. The method comprises the steps of a) applying a bacteria detection composition to an article, wherein the bacteria detection composition comprises a diatom and a sensor; b) placing the article near the product for a period of time; c) identifying a change in color of the sensor; and d) identifying the presence of the bacteria associated with the product, wherein the change in color of the sensor indicates the presence of the bacteria associated with the product. The previously described embodiments of the composition comprising a diatom and a sensor, of the bacteria detecting composition, and of the bacteria detecting system are applicable to the method of detecting a bacteria associated with a product described herein. In this embodiment, the product can be any product for which detection is desired, including food products, packages, luggage, clothing, air filters, envelopes, water, labels, wipes, and the like.

In another aspect of the present disclosure, a method of detecting an explosive degradation product is provided. The method comprises the steps of a) applying a explosive degradation product detection composition to an article, wherein the explosive degradation product detection composition comprises a diatom and a sensor; b) placing the article near a suspected explosive device for a period of time; c) identifying a change in color of the sensor; and d) identifying the presence of the explosive degradation product associated with the suspected explosive device, wherein the change in color of the sensor indicates the presence of the explosive degradation product associated with the suspected explosive device. The previously described embodiments of the composition comprising a diatom and a sensor, of the explosive degradation product detecting composition, and of the explosive degradation product detecting system are applicable to the method of detecting an explosive degradation product described herein.

The method of detecting an explosive degradation product includes the step of placing the article near a suspected explosive device. As used herein, the term “placing” means positioning the article in the vicinity of the suspected explosive device, for example in or on the suspected explosive device. In this embodiment, the product can be any product for which detection is desired, including food products, packages, luggage, clothing, air filters, envelopes, water, labels, wipes, and the like.

In another aspect of the present disclosure, a method of detecting a neurotoxin on a product is provided. The method comprises the steps of a) applying a neurotoxin detection composition to an article, wherein the neurotoxin detection composition comprises a diatom and a sensor; b) contacting the article and the product; c) identifying a change in color of the sensor; and d) identifying the presence of the neurotoxin on the product, wherein a lack of change in color of the sensor indicates the presence of the neurotoxin on the product. The previously described embodiments of the composition comprising a diatom and a sensor, of the neurotoxin detecting composition, and of the neurotoxin detecting system are applicable to the method of detecting a neurotoxin on a product described herein. In this embodiment, the product can be any product for which detection is desired, including food products, packages, luggage, clothing, air filters, envelopes, water, labels, wipes, and the like.

### EXAMPLE 1

### Preparation of Self-Assembled Nanostructured Compositions and Application to Articles

The nanostructured diatom sensor compositions of the present disclosure can be prepared and applied to various articles and be utilized for detection of gases, bacteria, explosive degradation products, and/or neurotoxins. In the various embodiments, the article to which the nanostructured diatom sensor composition is applied can be paper, packaging, a tissue towel, a nonwoven, a gas permeable coating, a gas permeable paint, a storage wall, a filter, a felt tip, or any object capable of application.

In this example, the self-assembled nanostructured compositions were prepared and applied to paper. Paper coating formulations can be optimized for sensitivity, selectivity and durability for the basic volatile compounds to be detected (for example, bacteria in various food packaging storage conditions). Optimization of paper coatings for sensor detection applications involves varying coating weight, binder type and viscosity, and cationic and wet strength additive usage. In addition, pH sensitive dyes, enzymes, and/or salts can be added in various portions to the diatom sensor compositions to determine the range suitable for the maximum sensitivity and selectivity.

The self-assembled nanostructured compositions were prepared and applied to paper by applying a coating formulation based on diatomite, polyvinyl alcohol (PVOH), pH sensitive dyes, and cationic additives mixture on acidic paper substrate. A diatomite dispersion in water (i.e., a diatomite slurry) was prepared using between 30-40% solids content via a high shear disperser under high agitation. Diatomites used in the coating formulation of this example had an average surface area of 30.1 m²/g.

PVOH was selected as a binder in the coating formulation of this example because of its high binding ability and high water absorption capacity. However, other polymer binders may be utilized as well. In this example, the solution of PVOH was prepared containing approximately 20% solids by adding the required amount of dry PVOH powder to cold water under agitation and heating the mixture to 85° C. A de-foaming composition was added before the addition of PVOH. The solution was held at approximately 85° C. for about 35-40 minutes to ensure substantially complete dissolution and hydration of PVOH.

The PVOH solution was then cooled to approximately 25° C. before it was added to the diatomite slurry at a pigment to binder ratio of 4:1 (pigment: 100 parts and binder: 25 parts) to 10:1 under slow agitation. Cationic additives such as Poly-DADMAC, polyvinylamine and polyethylenemine cationic additives can optionally be utilized to enhance the fixation and stability of the dye into the diatom/diatomites. The addition amounts of the optional cationic additives can be added at a ratio of about 0.5-2 parts.

Finally, pH sensitive dye were added into the coating formulation at a ratio of between 0.05 parts and 1 part. The coatings were mixed for about 20-30 minutes under low shear to ensure that no air is entrapped in the coating to cause foaming.

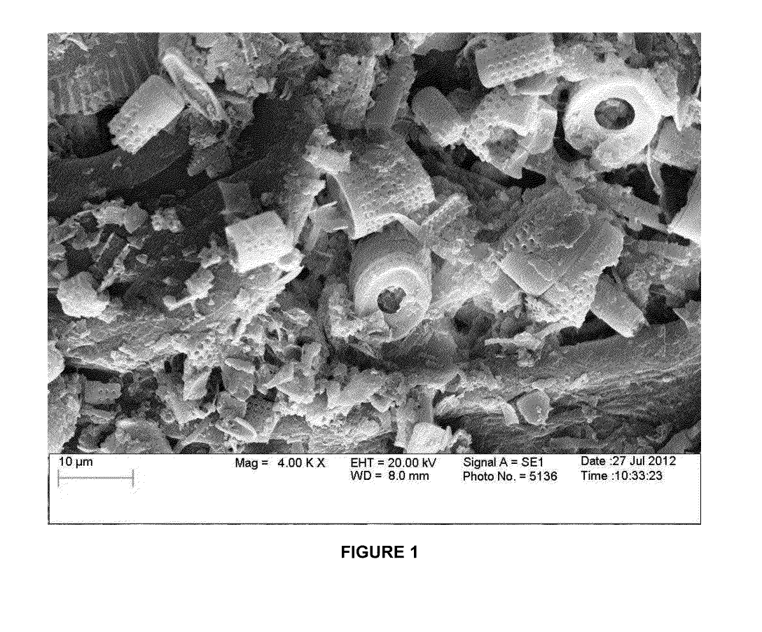

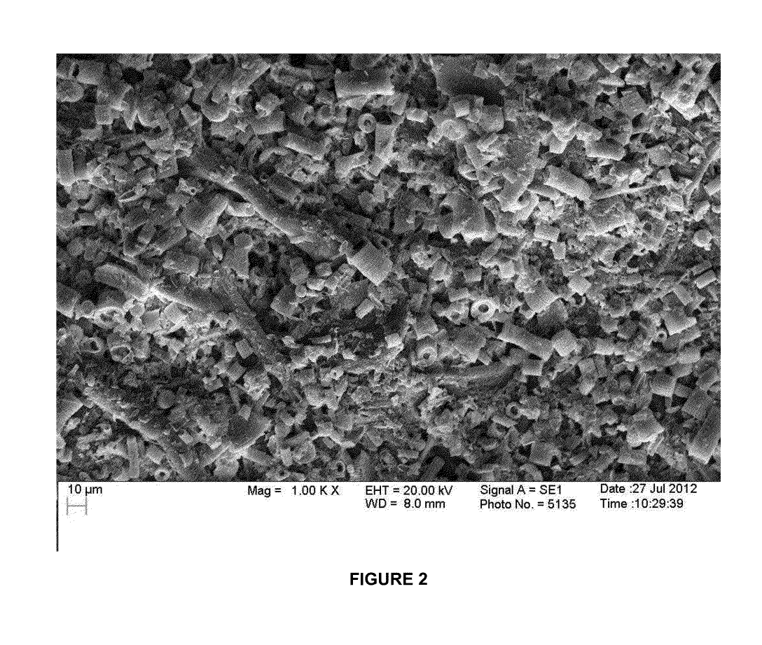

Application of the coatings on acidic paper was made by rods on drawdown platform. The coat weights applications varied by rod number and the solids content of the coating. The coat weights were applied to the paper between 5 and 20 g/m². FIG. 1 and FIG. 2 show the compositions comprising a diatom and a sensor as applied to the surface of paper.

### EXAMPLE 2

### Application of Self-Assembled Nanostructured Compositions to Articles

Other methods of applying the nanostructured diatom sensor compositions to paper are contemplated. Paper can be produced in handsheet molds. Rosin size and alum can be added into the papermaking furnish to acidify the paper and to reduce the migration of coating components into the paper. A slurry of diatoms can be prepared by dispersing the diatoms in water. In addition, an amount of binder (e.g., polyvinyl alcohol (PVOH) or latex binders such as high glass transition temperature latex binders) can be prepared. Cationic additives such as Poly-DADMAC, polyvinylamine, and polyethylenemine can be used to enhance the fixation and stability of the dye into the diatoms. Wet strength of the paper surface can be enhanced by additives such as glyoxal modified polyacrylamide or polyamide epichlorohydrine. If a meaningful level of dye leaching is found, ion pairing of the dyes with a quaternary ammonium salt such as cetyl trimethyl ammonium, bromide, octadecyl ammonium bromide, tetrahexyl ammonium bromide, and/or tetraoctyl ammonium bromide can be used to minimize dye leaching.

The coating formulation can be prepared by mixing amounts of the diatom slurry, binder functional additives, and pH sensitive dyes (e.g., bromocresol purple or bromocresol green). The dyes can either be mixed directly into the diatom slurry or into the coating color. The pH sensitive dyes can be added at a ration between about 0.125 parts to about 1 part.

The coating formulation comprising the dye can then be applied to the paper using a drawdown machine and a rod. Techniques for application using a drawdown machine and one or two rods are well known in the art. The applied coating on the paper is thereafter left to dry in the air or is dried using an air drying machine.

Alternatively, another approach for applying the nanostructured diatom sensor compositions to paper involves first applying a one-layer coating application on the paper, wherein the coating application contains diatoms, a binder (e.g., polyvinyl alcohol), and a low molecular weight cationic polymer (e.g., polyethylenimine). Thereafter, the required chemicals and enzyme are applied to the paper over the coated layer. This application can easily be achieved by both traditional paper coating and piezoelectric ink-jet printing techniques. Advantageously, this application does not require multiple “sandwich-type” layers on paper compared to other methods in the art.

Application techniques can be based on adsorption, absorption, electrostatic charge, and covalent attachment. Absorption and charge neutralization are dominant methods in pH sensitive ink immobilization for gas sensors. In case of enzymes and salts sensors, covalent bonding or physical encapsulation may be used.

In solvent formulation, where covalent binding techniques are utilized for application, silanized diatom particles in alcohol can be stirred and sonicated, then the suspension can be sprayed on paper manually (e.g., using a glass vaporizer). Many silanization procedures can be applied to diatoms. One such procedure involves placing diatom particles and dehydrated toluene in a flask followed by addition of dodecyltrichlorosilane in to the mixture, followed by refluxing and filtration.

### EXAMPLE 3

### Self-Assembled Nanostructured Compositions Identify Food Spoilage by Detecting Ammonia Gas

The nanostructured diatom sensor compositions of the present disclosure can be applied to various articles and be utilized for detection of gases, bacteria, explosive degradation products, and/or neurotoxins. In this example, the nanostructured diatom sensor compositions were applied to paper and evaluated for identification of food spoilage by detecting ammonia gas. The application to paper was as described in Example 2.

The paper-based diatom sensors with different coating formulations were first evaluated at various levels of volatile ammonia. Four different coating formulations were applied to the paper-based diatom sensors in the various tubes: (a) the coating formulation was applied at 6 g/m² coat weight and contained 0.5 parts of dye; (b) the coating formulation was applied at 20 g/m² coat weight and contained 0.5 parts of dye; (c) the coating formulation was applied at 6 g/m² coat weight and contained 1 part of dye; (d) the coating formulation was applied at 20 g/m² coat weight and contained 1 part of dye.

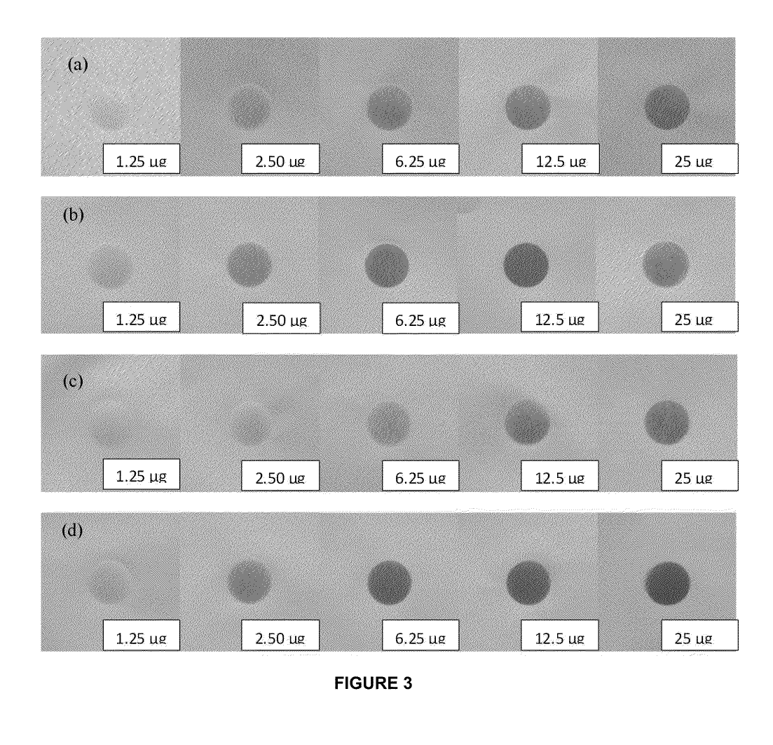

FIG. 3 shows the evaluated response of the paper-based diatom sensors for each coating formulation. Each formulation was evaluated at five different levels of volatile ammonia: 1.25 μg, 2.50 μg, 6.25 μg, 12.5 μg, and 25 μg. As shown in FIG. 3, the response of the sensors increased with the increased amount of volatile ammonia for each coating formulation.

In addition, Nile tilapia and channel catfish were used to test the identification of food spoilage using nanostructured diatom sensors applied to paper. Approximately 10 gram samples of tilapia and of catfish were placed into tubes containing the paper-based diatom sensors and were stored at 4° C. Microbial analysis and identification were subsequently performed.

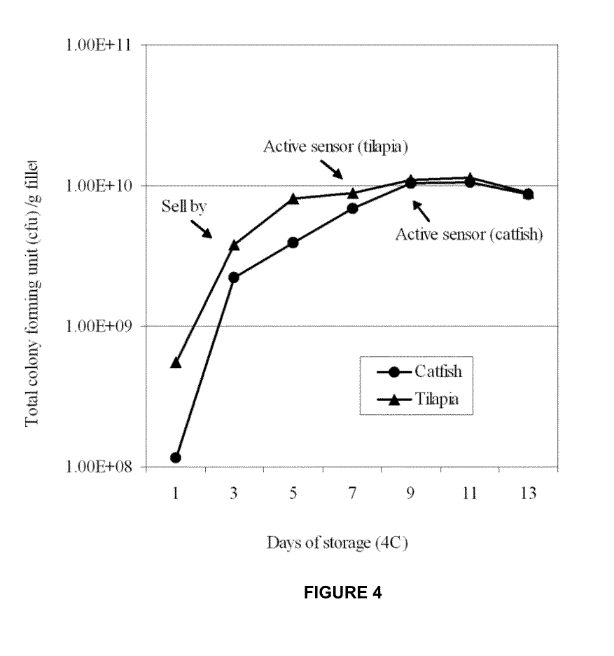

For both tilapia and catfish, the total bacterial colony forming units (CFU) were calculated for each day the samples were in storage using a standard soy agar plate count. As shown in FIG. 4, the “sell by” date of the tilapia and the catfish occurred on Day 3 of storage. The diatom sensors were activated (i.e., achieved coloration) on Day 7 of storage (for tilapia) and on Day 9 of storage (for catfish), which correlated to a CFU of approximately 1.0×10¹⁰ for each fish. The activation of the diatom sensors unexpectedly correlated at the same level of CFU for each type of fish. Furthermore, the diatom sensors unexpectedly detected basic nitrogen compounds (e.g., ammonia) at an order of 10⁻² micrograms, corresponding to approximately 5 parts per million (ppm). This sensitivity is approximately three orders of magnitude greater than the sensitivity findings generated by other researchers, and allows for surprisingly high levels of sensitivity compared to those known in the art.

### EXAMPLE 4

### Additional Food Spoilage Identification Using Self-Assembled Nanostructured Compositions

Additional experiments of other fish and poultry spoilage using the nanostructured diatom sensors can be performed. For example, experiments can be done at selected temperatures with cold storage (4° C.) and temperature-abused display conditions (9° C.). Meat samples for microbiological analyses can be prepared by mixing 10 g of meat product in 90 ml of 0.85% phosphate buffered saline (PBS, pH 7.2). A tenfold serial dilutions of each sample (in duplicate) can then be prepared in PBS and 0.1 mL of serially diluted samples can be plated onto pseudomonas plate. After 2 days of growth at 26° C., the total viable colonies can be counted and recorded to correlate with the sensor response.

Since the surface of meat is the most vulnerable to contaminated with bacteria, detection and enumeration of bacterial contamination on the surface area by a swab test can be performed to investigate and determine the sensitivity of the diatom sensors and their feasibility as commercial food packaging materials. Along with conventional microbiological assays, a 3M Petrifilm Aerobic Count Plate and an E. coli/Coliform Count method can also be performed. An ammonia ISE meter can be used to measure the ammonia content in the meat products and general pH measurements for coatings and ink-jet fluids utilizing the diatom sensors.

### EXAMPLE 5

### Detection of Bacteria Using Self-Assembled Nanostructured Compositions

The nanostructured diatom sensor compositions of the present disclosure can be utilized for detection of bacteria in or on a product. For example, detection of Escherichia coli (E. coli) can be performed using the diatom sensors.

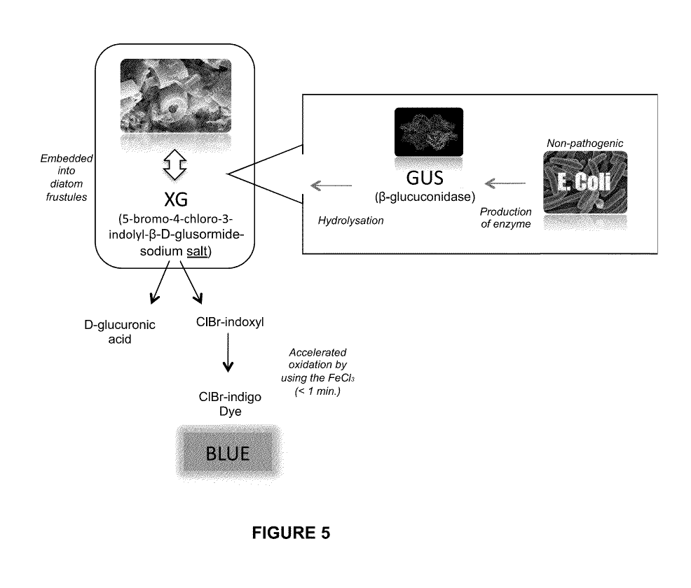

One exemplary embodiment of diatom sensors can be formulated for the detection of E. coli using a salt as the sensor that is incorporated into the diatom. FIG. 5 shows the mechanism by which such embodiments may be made by incorporating XG salts (aka 5-bromo-4-chloro-3-indolyl-β-D-glusormide-sodium salts) as a sensor in the diatoms.

It is known that E. coli bacteria produce the enzyme β-Glucuronidase (GUS). After GUS is expressed by E. coli, GUS is hydrolyzed in the presence of XG salts incorporated in the diatoms. Thereafter, a chemical reaction causes the sensor to turn blue, indicating presence of E. coli in or on the product (see FIG. 5).

### EXAMPLE 6

### Detection of Neurotoxins Using Self-Assembled Nanostructured Compositions

The nanostructured diatom sensor compositions of the present disclosure can be utilized for detection of a neurotoxin in or on a product. For example, detection of acetylcholinesterase (AChE) can be performed using the diatom sensors.

One exemplary embodiment of diatom sensors can be formulated for the detection of acetylcholinesterase incorporates acetylthiocholine (ATCh) and dithiobisnitrobenzoate (DTNB) into the diatom. The diatoms may be applied to an article, such as paper, for the detection of AChE enzyme.

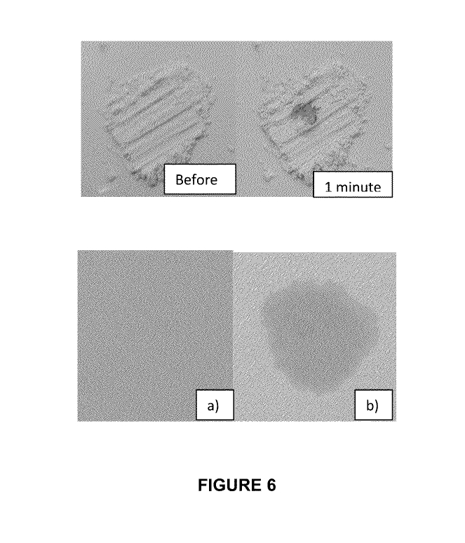

The top panel of FIG. 6 shows an example in which AChE enzyme was detected using the diatom sensors. 4 μl of AChE enzyme was applied on a bed of diatomites treated with a mixture of ATCh and DTNB. Detection was performed on ceramic tile after one minute (right box) and demonstrated identification of AChE enzyme.

The bottom panel of FIG. 6 shows an example in which AChE enzyme was detected using the diatom applied to paper. Box (a) shows AChE enzyme immobilized on paper, but with no ATCh or DTNB incorporated into the diatoms. Box (b) shows AChE enzyme immobilized on paper, with ATCh (2 μI) and DTNB (2 μI) incorporated into the diatoms. AChE enzyme was identified in the Box (b) embodiment, where ATCh and DTNB were incorporated into the diatoms.

### EXAMPLE 7

### Detection of Neurotoxins Using Self-Assembled Nanostructured Compositions Via Counter Reaction

Another example of detecting AChE using the nanostructured diatom sensor compositions is based on the appearance of distinct color changes on paper substrate when neurotoxins are not present (i.e., a counter reaction) and is based on Ellman's assay.

AChE inhibitors are known neurotoxins, and are used as the exemplary neurotoxin in this example. The diatoms may be formulated to incorporate ATCh and DTNB. It is known that ATCh reacts with AChE (i.e., a strong hydrolyzer) to produce thiocholine. This reaction can then cause thiocholine to react with DTNB to generate 5-thio-2-nitrobenzoate, which is yellow in color. As a result, the diatom sensor changes to a yellow color.

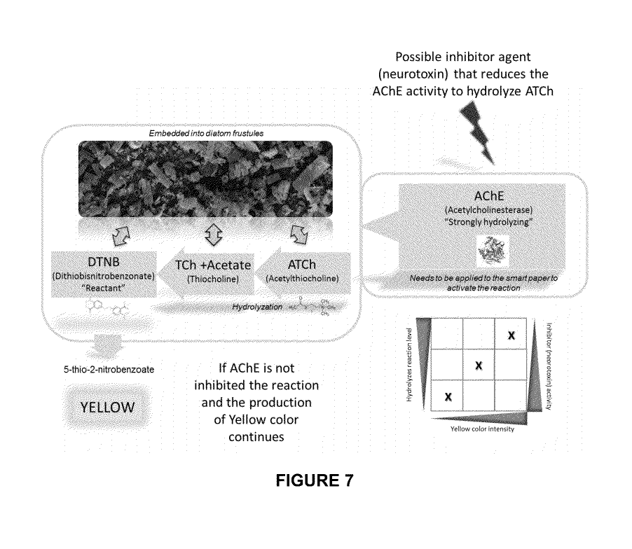

An AChE inhibitor (i.e., a neurotoxin) will inhibit AChE activity. Thus, if an AChE inhibitor is present on a product, AChE activity will be suppressed and the hydrolyzation reaction described above will not occur. As a result, the diatom sensor does not change to the yellow color. The absence of the color yellow indicates detection of an AChE inhibitor (i.e., a neurotoxin) on the product. FIG. 7 shows the mechanism of action for the embodiment of this example.

### EXAMPLE 8

### Detection of Neurotoxins Using Self-Assembled Nanostructured Compositions Via Counter Reaction in a Pen-Like Device

The embodiment described in Example 7 can be incorporated into a pen-like device embodiment for rapid and efficient detection of neurotoxins using the nanostructured diatom sensor compositions. In the present example, the nanostructured diatom sensor compositions can be applied to a felt tip that can be applied to the tip of a pen-like device. The felt tip can comprise diatoms incorporating ATCh and DTNB as described above.

The pen-like device can also comprise an ampoule with dual compartments. One compartment of the ampoule can contain acetylcholinesterase (AChE) enzyme. The AChE enzyme can be a lyophilized (freeze dried) enzyme in dry form. This can allow the AChE enzyme to be preserved for a longer period of time. A second compartment of the ampoule can contain water.

The pen-like device could be activated by clicking a functional moving puncture needle in order to break the contents of the ampoule. This can release the resulting AChE solution to be channeled to the tip of the pen-like device on which the felt tip (i.e., comprising diatoms incorporating ATCh and DTNB) is applied.

After the pen-like device is activated, a product of interest can be investigated for the presence of neurotoxins (e.g., AChE inhibitors). The surface of the product can be wiped with the felt tip of the pen-like device. As described in Example 7, if no neurotoxin is present on the product, the felt tip will change color to yellow (i.e., the hydrolysis reaction takes place). However, when a neurotoxin is present on the product, the felt tip will not change color to yellow (it may either remain white in color, or change to a lesser tone of yellow).

The felt tip of the pen-like device may be exchangeable, and could be replaced with each test of product.

The embodiment in this example is advantageous compared to known processes in the art. In this embodiment, the ATCh and AChE components can be present in forms in which they may remain stable for longer periods of time. In comparison, ATCh and AChE components known in the art are very sensitive to degradation and may only be stable for approximately one hour for the detection of neurotoxins.

Alternatively, the pen-like device could be formulated by placing ATCh in the ampoule and applying AChE to the felt tip.

While the invention has been illustrated and described in detail in the foregoing description, such an illustration and description is to be considered as exemplary and not restrictive in character, it being understood that only the illustrative embodiments have been described and that all changes and modifications that come within the scope of the invention are desired to be protected. Those of ordinary skill in the art may readily devise their own implementations that incorporate one or more of the features described herein, and thus fall within the scope of the present invention.

What is claimed is:

1. A composition comprising a diatom and a sensor, wherein the surface of the diatom comprises one or more pores, and wherein at least part of the sensor is contained within one or more pores in the diatom.

2. The composition of claim 1, wherein the sensor is a dye.

3. The composition of claim 1, wherein the sensor changes color following exposure to a gas.

4. The composition of claim 3, wherein the gas is a total volatile basic nitrogen (TVB-N) gas.

5. The composition of claim 3, wherein the gas is ammonia.

6. The composition of claim 1, wherein the sensor changes color following exposure to volatile basic nitrogen.

7. The composition of claim 1, wherein the composition further comprises an article, and wherein the diatom is applied to the article.

8. The composition of claim 7, wherein the diatom is applied to the article using a polymer binder.

9. The composition of claim 7, wherein the article is paper.

10. The composition of claim 9, wherein the diatom is applied to the paper using inkjet printing.

11. The composition of claim 7, wherein the paper is a sticker.

12. The composition of claim 7, wherein the paper is a label.

13. A method of identifying the presence of a gas associated with a food product, said method comprising the steps of
a) applying a gas detection composition to an article, wherein the gas detection composition comprises a diatom and a sensor;
b) placing the article near the food product for a period of time;
c) identifying a change in color of the sensor; and
d) identifying the presence of the gas associated with the food product, wherein the change in color of the sensor indicates the presence of the gas associated with the food product.

14. The method of claim 13, wherein the sensor is a dye.

15. The method of claim 13, wherein the gas is a total volatile basic nitrogen (TVB-N) gas.

16. The method of claim 13, wherein the gas is ammonia.

17. The method of claim 13, wherein the sensor changes color following exposure to volatile basic nitrogen.

18. The method of claim 13, wherein the gas detecting composition is applied to the article using a polymer binder.

19. The method of claim 13, wherein the article is paper.

20. The method of claim 13, wherein the identification of the presence of the gas associated with the food product has a sensitivity of about 5 parts per million.
